(19) United States
(12) Patent Application Publication
(10) Pub. No.: US 2022/0273620 A1
(43) Pub. Date: Sep. 1, 2022

(54) COMPOSITIONS CONTAINING TOAD SECRETION COMPOUNDS

(71) Applicant: CAAMTECH, INC., Issaquah, WA (US)

(72) Inventor: Andrew R. CHADEAYNE, Issaquah, WA (US)

(21) Appl. No.: 17/627,988

(22) PCT Filed: Jul. 23, 2020
(86) PCT No.: PCT/US2020/043204

§ 371 Date: Jan. 18, 2022

Related U.S. Application Data
(60) Provisional application No. 62/877,629, filed on Jul. 23, 2019.
(60) Provisional application No. 62/877,639, filed on Jul. 23, 2019.
(60) Provisional application No. 62/877,619, filed on Jul. 23, 2019.
(60) Provisional application No. 62/877,668, filed on Jul. 23, 2019.
(60) Provisional application No. 62/877,642, filed on Jul. 23, 2019.
(60) Provisional application No. 62/877,664, filed on Jul. 23, 2019.
(60) Provisional application No. 62/877,674, filed on Jul. 23, 2019.
(60) Provisional application No. 62/877,649, filed on Jul. 23, 2019.
(60) Provisional application No. 62/877,659, filed on Jul. 23, 2019.
(60) Provisional application No. 62/877,635, filed on Jul. 23, 2019.
(60) Provisional application No. 62/877,628, filed on Jul. 23, 2019.
(60) Provisional application No. 62/877,662, filed on Jul. 23, 2019.
(60) Provisional application No. 62/877,625, filed on Jul. 23, 2019.
(60) Provisional application No. 62/877,672, filed on Jul. 23, 2019.
(60) Provisional application No. 62/877,648, filed on Jul. 23, 2019.
(60) Provisional application No. 62/877,653, filed on Jul. 23, 2019.
(60) Provisional application No. 62/877,634, filed on Jul. 23, 2019.
(60) Provisional application No. 62/877,618, filed on Jul. 23, 2019.
(60) Provisional application No. 62/877,654, filed on Jul. 23, 2019.
(60) Provisional application No. 62/877,645, filed on Jul. 23, 2019.
(60) Provisional application No. 62/877,658, filed on Jul. 23, 2019.
(60) Provisional application No. 62/877,651, filed on Jul. 23, 2019.
(60) Provisional application No. 62/877,643, filed on Jul. 23, 2019.

(51) Int. Cl. A61K 31/4045; A61K 31/675; A61K 35/65

(52) U.S. Cl. CPC A61K 31/4045; A61K 31/675; A61K 35/65

(57) ABSTRACT

Disclosed are compositions containing two purified toad secretion tryptamines chosen from the following 5-MeO-DMT, 5-MeO-NMT, 5-Methoxytryptamine, bufobutanoic Acid, bufobutarginine, bufoserotonin A, bufoserotonin B, bufoserotonin C, bufotenidine, bufotenin, bufotenin Oxide, bufotenine-O-Sulphate, bufoviridine, dET, dMT, n-Acetylserotonin, n′-Formylserotonin, n-Methylserotonin, o-Methylbufoviridine, serotonin, tryptamine, and bufopyramide or the salts of these toad secretion tryptamines. The disclosure also relates to formulations, including pharmaceutical formulations, of such a composition and an excipient. Pharmaceutical formulation further containing a therapeutically effective amount of a serotonergic drug, a purified psilocybin derivative, a purified cannabinoid, or a purified terpene a disclosed. Also disclosed are methods of regulating the activity of a neurotransmitter receptor and methods of treating a psychological disorder, a compulsive disorder, or a depressive disorder.

### CROSS-REFERENCE TO RELATED APPLICATIONS

This application claims priority to U.S. application Nos. 62/877,618; 62/877,619; 62/877,625; 62/877,628; 62/877,629; 62/877,634; 62/877,635; 62/877,639; 62/877,642; 62/877,643; 62/877,645; 62/877,648; 62/877,649; 62/877,651; 62/877,653; 62/877,654; 62/877,658; 62/877,659; 62/877,662; 62/877,664; 62/877,668; 62/877,672; 62/877,674; all filed Jul. 23, 2019, the disclosures of which are each incorporated by reference.

### TECHNICAL FIELD

The disclosure relates to new compositions and formulations purified toad secretion tryptamines as well as methods for the therapeutic use of those compositions and formulations.

### BACKGROUND

Several genera of toads secrete chemicals (i.e. toad secretions, aka venoms) from the parotid glands in their skin that are located on their back, neck, and shoulders. Two of the largest and most well-studied genera of toads are Bufo and Rhinella.¹ Toad secretions can be highly toxic. They are used as a defense mechanism for repelling potential predators. The secretions also protect the toad's skin against the growth of fungi, bacteria, and protozoa.² For centuries, South American cultures have used toad and frog secretions as components in poison arrows and darts for hunting. Since ancient times, Chinese folk medicine has used toad secretions as a local anesthetic and for treating a variety of conditions, including inflammation and cancer.

Researchers have identified numerous compounds in toad secretions including tryptamines, indole alkaloids, steroids, organic acids, and tryptophols, some of which have psychedelic effects. These compounds include 5-MeO-DMT (5-methoxy-N,N-dimethyltryptamine), bufotenin, and DMT (N,N-dimethyltryptamine).³ Toad secretions also contain compounds such as N-methylserotonin, bufogenin, 5-methoxytrytophol, bufoviridine, 5-hydroxytryptophol, 5-hydroxyindoleacetic acid, 5-methoxytryptophol, and 5-methoxyindoleacetic acid.^{3,4} In the United States, toad secretions are often used recreationally and many of the compounds therein are classified as Schedule I drugs under the Controlled Substances Act.

The skin secretions of Bufo alvarius are composed primarily of the psychoactive compound 5-MeO-DMT which was first isolated in 1959 from the bark of the plant Dictyoloma incanescens.⁵ The levels of 5-MeO-DMT in toad secretions can be as high as 15%.⁴ 5-MeO-DMT elicits its psychedelic effects via G protein-coupled receptor pathways. Research has shown that 5-MeO-DMT is an agonist primarily of the serotonin 5-HT_{2A} receptor as well as 5-HT_{2C} and 5-H_{1A}.⁶

Prior to 1994, only plant- and fungi-based sources of psychoactive compounds were known. There was no scientific documentation of psychedelic agents in the animal kingdom other than a few scattered reports. Then, in 1994, a study published in the Journal of Ethnopharmacology revealed that the secretions of the Colorado River toad Bufo alvarius were a powerful entheogen when dried and smoked.⁷ This discovery not only revolutionized the understanding of psychedelic compounds in the animal kingdom, it changed the view that historians and anthropologists had of pre-Columbian people who lived in proximity to B. alvarius and other toads. It has long been theorized that these people used psychoactive plants to make their ritual psychoactive preparations. But this new evidence suggested they probably used toad secretions.

Another component of toad secretions, DMT, is also one of the primary active ingredients of ayahuasca, a cocktail of plant compounds used in religious ceremonies by South American tribes.⁸ It was first synthesized by Manske in 1931,⁹ and Szàra discovered its psychedelic properties in 1956.¹⁰

Bufotenin was discovered in 1920 by Hans Handovsky at the University of Prague.¹¹ He isolated bufotenin along with several known compounds from the skin of B. vulgaris. Wieland et al. confirmed the chemical structure of bufotenin in 1934¹² and Hoshino and Shimodaria first synthesized it in 1935.¹³ Since then, researchers have also found bufotenin in the retina and brain tissue of toads.¹⁴ In addition to toads, bufotenin has also been found in frogs, mushrooms, higher plants, and mammals. Interestingly, people with schizophrenia often have bufotenin in their brain, plasma, and urine.¹⁵⁻¹⁷

In humans, bufotenin is a potent agonist of the serotonin 5-HT2A receptor, which accounts for its psychoactive properties.¹⁸ Historically, the receptor function of bufotenin has been more well-studied in rats and mice than in humans. In addition to 5-HT_{2A}, researchers have found potent binding activity of bufotenin in several serotonin receptors in the rat brain cortex including 5-HT_{1A} and 5-HT_{1B}¹⁹ and 5-HT₃.²⁰ Bufotenin is also a potent agonist of 5-HT_{1A} receptors in the rat brain hippocampus.²¹

Some compounds found in toad secretions are being studied to identify their possible therapeutic benefits. For example, studies are finding that 5-MeO-DMT improves symptoms of anxiety and depression^{3,22} as does DMT.²³

There is an unmet need for purposefully formulated products containing specific compositions of single isolated compounds purified from toad secretions. For example, there is a need for formulations wherein each purified toad secretion compound, one separated from the animal's secretion within which it existed in nature, is purposefully formulated to optimize its beneficial effects when administered to subject.

### SUMMARY

The disclosure relates to compositions comprising, consisting essentially of, or consisting of two purified toad secretion tryptamines chosen from the following 5-MeO-DMT, 5-MeO-NMT, 5-Methoxytryptamine, bufobutanoic Acid, bufobutarginine, bufoserotonin A, bufoserotonin B, bufoserotonin C, bufotenidine, bufotenin, bufotenin Oxide, bufotenine-O-Sulphate, bufoviridine, dET, dMT, n-Acetylserotonin, n′-Formylserotonin, n-Methylserotonin, o-Methylbufoviridine, serotonin, tryptamine, and bufopyramide or the salts of these toad secretion tryptamines. In a composition of the disclosure the molar ratio of the first purified toad secretion tryptamine to the second toad secretion tryptamine in the composition is from 0.1:100 to 100:01, from 1:100 to 100:1, from 1:50 to 50:1, from 1:25 to 25:1, from 1:20 to 20:1, from 1:10 to 10:1, from 1:5 to 5:1, from 1:2 to 2:1 or is 1:1.

The disclosure also relates to formulations comprising a composition of comprising, consisting essentially of, or consisting of two purified toad secretion tryptamines and an excipient. The formulation may be a pharmaceutical formulation comprising a composition of the disclosure and a pharmaceutically acceptable excipient, where the first purified toad secretion tryptamine and the second toad secretion tryptamine are each present in a therapeutically effective amount. In one embodiment a pharmaceutical formulation may further comprise a therapeutically effective amount of a serotonergic drug, a purified psilocybin derivative, a purified cannabinoid, or a purified terpene.

Also disclosed are methods of regulating the activity of a neurotransmitter receptor by administering to a person in need thereof an effective dose of a composition of the disclosure or of a formulation of the disclosure or by administering to a person in need thereof a pharmaceutical formulation of the disclosure.

The disclosure also provides methods of treating a psychological disorder, a compulsive disorder, or a depressive disorder by administering to a person in need thereof an effective dose of a composition of the disclosure or of a formulation of the disclosure or by administering to a person in need thereof a pharmaceutical formulation of the disclosure.

### DETAILED DESCRIPTION

Disclosed herein are new compositions and formulations purified toad secretion tryptamines as well as methods for the therapeutic use of those compositions and formulations. Non-naturally occurring toad secretion tryptamine compositions and formulations are disclosed. In each the toad secretion tryptamines are combined into compositions and formulations via human ingenuity to arrive at compositions and formulations that are not found in nature. These compositions and formulations have different physical properties from those found in nature and provide different pharmacological properties. In many cases, the disclosed compositions and formulations provide different clinical effects when administered to a subject.

This disclosure provides a composition comprising, consisting essentially of, or consisting of a first purified toad secretion tryptamine and a second purified toad secretion tryptamine. The first purified toad secretion tryptamine is different from the second toad secretion tryptamine meaning that the toad secretion tryptamine molecule of the “first purified toad secretion tryptamine” is structurally different from that of the “second purified toad secretion tryptamine.” Such structural differences, could be determined and demonstrated by a variety of analytical methods known in the chemical arts, including but not limited to chromatography, NMR, x-ray crystallography, etc. A composition of the disclosure may prepared using techniques known in the art such as mixing of a first purified toad secretion tryptamine and a second purified toad secretion tryptamine, forming a solution or slurry of a first purified toad secretion tryptamine and a second purified toad secretion tryptamine followed by solvent removal and other such techniques.

A composition of the disclosure comprises, consists essentially of or consists of two purified toad secretion tryptamines chosen from the following 5-MeO-DMT, 5-MeO-NMT, 5-Methoxytryptamine, bufobutanoic Acid, bufobutarginine, bufoserotonin A, bufoserotonin B, bufoserotonin C, bufotenidine, bufotenin, bufotenin Oxide, bufotenine-O-Sulphate, bufoviridine, dET, dMT, n-Acetylserotonin, n′-Formylserotonin, n-Methylserotonin, o-Methylbufoviridine, serotonin, tryptamine, and bufopyramide or the salts of these toad secretion tryptamines. The structural formulas of each of these toad secretion tryptamine compounds is shown below. Each is naturally present in many naturally occurring organisms, including Bufo olvarius, and occurs as a minor component within a mixture of many other molecules.

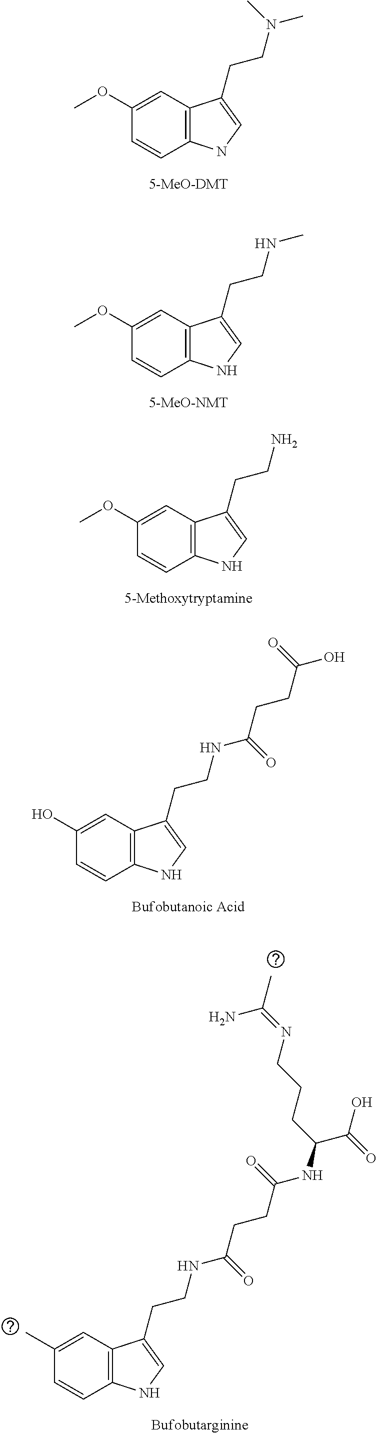

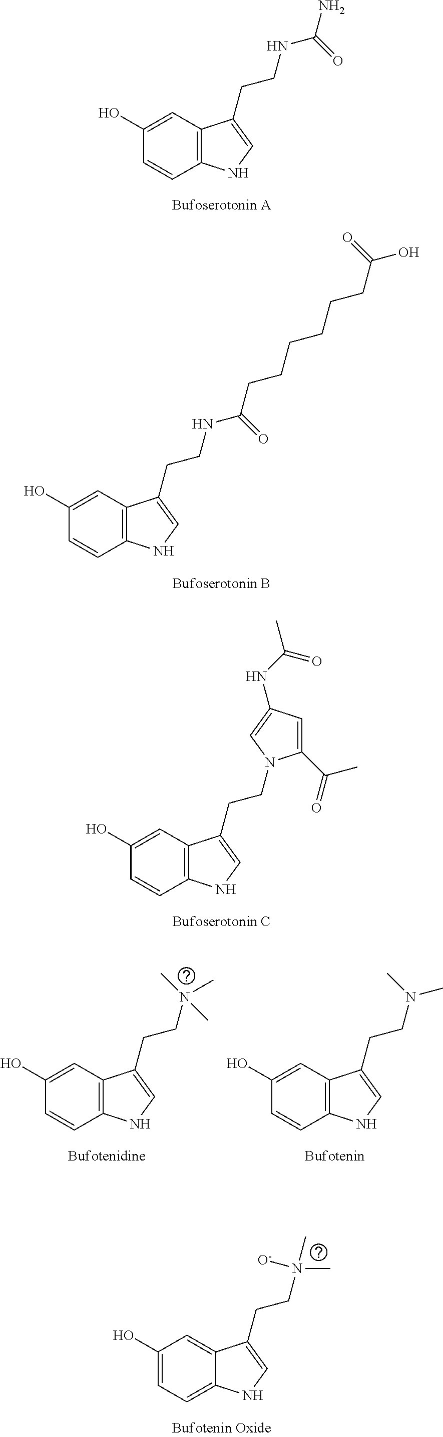

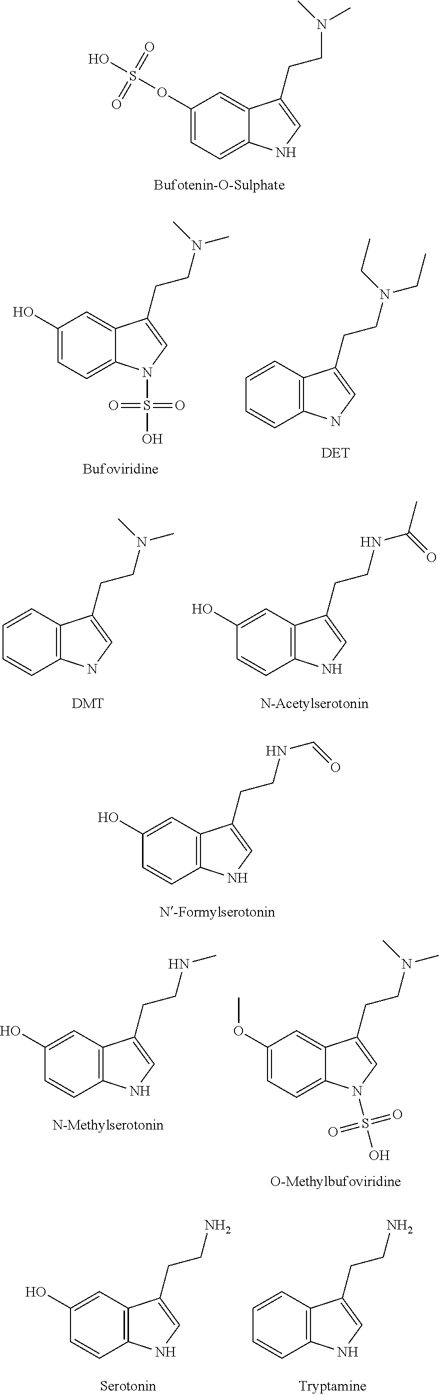

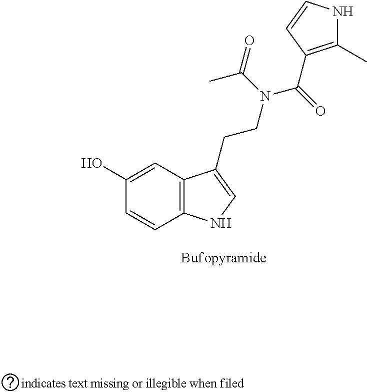 [SMILES: CC(=O)N(CCc1c[nH]c2ccc(O)cc12)C(=O)c1cc[nH]c1C]

Each of these compounds is naturally present in many naturally occurring organisms. 5-MeO-DMT, 5-MeO-NMT, 5-Methoxytryptamine, DET, DMT, and O-Methylbufoviridine naturally occur in Bufo alvarius. Bufobutanoic acid, Bufoserotonin A, Bufoserotonin B, Bufoserotonin C, Bufotenin Oxide, N-Acetylserotonin, N′-Formylserotonin, and Tryptamine are naturally present in bufo spp. Bufobutarginine is naturally present in many naturally occurring organisms, including Bufo gargarizans Cantor, and Bufo melanostictus Schneider. Bufotenidine is naturally present in Bufo americanus, Bufo arenarum, Bufo boreas boreas, Bufo boreas halophilus, Bufo bufo, Bufo bufo spinosus, Melanophryniscus moreirae, Rhinella schneideri, and Scaphiopus hammondi. Bufotenin is naturally present in Bufo alvarius, Bufo americanus, Bufo arenarum, Bufo boreas halophilus, Bufo bufo, Bufo bufo spinosus, Melanophryniscus moreirae, Rhinella schneideri, and Scaphiopus hammondi. Bufotenine-O-Sulphate is naturally present in Bufo bocourti, Bufo marinus, Bufo marmoreus, Bufo perplexus, and Bufo spinulosus chilensis. Bufoviridine is naturally present in Bufo alvarius, Bufo bocourti, Bufo bufo spinosus, and Bufo viridis. N-Methylserotonin is naturally present in Bufo alvarius, Bufo americanus, Bufo arenarum, Bufo boreas boreas, Bufo bufo, and Bufo bufo spinosus. Serotonin is naturally present in Bombina bombina, Bufo alvarius, Bufo americanus, Bufo arenarum, Bufo bergei, Bufo bufo, Bufo bufo spinosus, Bufo marinus, Melanophryniscus moreirae, Scaphiopus couchi, and Scaphiopus hammondi. Bufopyramide is naturally present in Bufo gargarizans Cantor, and Bufo melanostictus Schneider.

A composition of the disclosure contains a first purified toad secretion tryptamine and a second purified toad secretion tryptamine, wherein the first purified toad secretion tryptamine is different from the second purified toad secretion tryptamine. A composition of the disclosure comprises, consists essentially of or consists of two purified toad secretion tryptamines chosen from the following 5-MeO-DMT, 5-MeO-NMT, 5-Methoxytryptamine, bufobutanoic Acid, bufobutarginine, bufoserotonin A, bufoserotonin B, bufoserotonin C, bufotenidine, bufotenin, bufotenin Oxide, bufotenine-O-Sulphate, bufoviridine, dET, dMT, n-Acetylserotonin, n′-Formylserotonin, n-Methylserotonin, o-Methylbufoviridine, serotonin, tryptamine, and bufopyramide or the salts of these toad secretion tryptamines. Each separate composition of two of these purified toad secretion tryptamines or a salt thereof is a separate embodiment of the disclosure. The composition comprises, consisting essentially or consists of purposefully chosen amounts of at least two of these purified toad secretion tryptamines or a salt thereof. A purified toad secretion tryptamine may be prepared by extracting a desired tryptamine from its naturally occurring source or by chemical synthesis. A composition of the invention may be made by removing one or more tryptamines and other compounds from a naturally occurring source such that the resulting non-natural composition contains the desired tryptamines, those that remain after other tryptamines and compounds are removed. For example, bufotenidine may be removed from a toad secretion to prepare a composition of the disclosure.

Exemplary molar ratios of the first purified toad secretion tryptamine to the second toad secretion tryptamine in a composition of the disclosure include but are not limited to from about 0.1:100 to about 100:01, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1. Where a purposefully chosen composition contains more than two toad secretion tryptamines each toad secretion tryptamine within the purposefully chosen composition will be present in a molar ratio such as just mentioned with respect to each other toad secretion tryptamine present. A composition containing more than two purified toad secretion tryptamines may then be considered as a composition or mixture of first and second toad purified secretion tryptamines. For example, a composition may include a first purified toad secretion tryptamine, a second purified toad secretion tryptamine and a third purified toad secretion tryptamine selected from the same tryptamines as the first and second, wherein the first purified toad secretion tryptamine is different from each of the second purified toad secretion tryptamine and the third toad secretion tryptamine. Or, a composition may include a first purified toad secretion tryptamine, a second purified toad secretion tryptamine and a third and a fourth purified toad secretion tryptamine selected from the same tryptamines as the first and second, wherein the first purified toad secretion tryptamine is different from each of the second purified toad secretion tryptamine and the third and fourth toad secretion tryptamine.

In one embodiment of a composition of the disclosure the first purified toad secretion tryptamine is 5-MeO-DMT, 5-MeO-NMT, 5-Methoxytryptamine, bufotenidine, dET or dMt. Or, in other scompositions of the disclosure the first purified toad secretion tryptamine is 5-MeO-DMT, 5-MeO-NMT, or 5-Methoxytryptamine. In another embodiment the first purified toad secretion tryptamine and the second purified tryptamine is selected from 5-MeO-DMT, 5-MeO-NMT, 5-Methoxytryptamine, bufotenidine, dET or dMt.

Disclosed herein are formulations, such as pharmaceutical formulations, utilizing a purposefully chosen composition of two or more individual purified toad secretion tryptamines and optionally an inactive compound, such as an excipient. A pharmaceutical formulation contains a therapeutically effective amount of each individual purified toad secretion tryptamine. A “therapeutically effective amount of each individual purified toad secretion tryptamine is an amount which correlates to a therapeutic effect and may separately range from, for example, about 0.5-about 200 mg, about 1 mg-about 100 mg, about 2 mg-about 50 mg, about 5 mg-about 25 mg or 25 mg. The actual amount required for treatment of any particular disease, disorder or condition for any particular patient may depend upon a variety of factors including, for example, the particular disease, disorder or condition being treated; the disease state being treated and its severity; the specific pharmaceutical composition employed; the age, body weight, general health, sex and diet of the patient; the mode of administration; the time of administration; the route of administration; and the rate of excretion; the duration of the treatment; any drugs used in combination or coincidental with the specific compound employed; and other such factors well known in the medical arts. The total amount of purified toad secretion tryptamines in a formulation may range from about 0.01 to 100 wt. %, from about 0.1 to 100 wt. %, from about 1 to about 99 wt. %, from about 50 to about 90 wt. %, from about 5 to about 75 wt. %, from about 10 to about 50 wt. %, from about 10 to about 25 wt. %, or from about 15 to about 40 wt. %.

A composition of the invention may be formulated in any type or pharmaceutical formulation known in the art. A pharmaceutical formulation of the disclosure may be solid dosage form such as an oral dosage form, e.g. a pill, capsule, and the like, which may or may not be enterically coated. A composition of the disclosure may also be formulated as a pharmaceutical formulation designed to avoid first-pass metabolism. Accordingly, to avoid first pass metabolism, a composition of the invention may be formulated as a transdermal formulation, a sublingual formulation, a buccal formulation, an intravenous (I.V.) formulation, a subcutaneous (S.C.) formulation or an inhalation formulation.

A formulation of a composition of the disclosure may contain additional inactive compounds, such as excipients, binders, stabilizers, permeation enhancers, solubilizers, etc. as known in the art. For example in a pharmaceutical formulation a composition of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents.

A pharmaceutical formulation of a composition of the disclosure may contain additional active compounds found within one or more of the following organisms: Bufo alvarius, bufo spp., Bufo gargarizans Cantor, Bufo melanostictus Schneider, Bufo americanus, Bufo arenarum, Bufo boreas boreas, Bufo boreas halophilus, Bufo bufo, Bufo bufo spinosus, Melanophryniscus moreirae, Rhinella schneideri, Scaphiopus hammondi, Bufo bocourti, Bufo marinus, Bufo marmoreus, Bufo perplexus, Bufo spinulosus chilensis, Bufo viridis, Bombina bombina, Bufo bergei, and Scaphiopus couchi. In one embodiment the disclosed formulations comprise additional active compounds not found within these organisms.

A pharmaceutical formulation of the disclosure may contain an additional active compound selected from the group consisting of: [2M-H] adipyl arginine, [2M-H] pimeloyl arginine, [2M-H] suberoyl arginine, 1-beta-hydroxybufalin, 1-methyl-2,9-dihydro-1H-pyrido[3-b]indol-6-ol, 11α-hydroxy hellebrigenin, 11α-hydroxymarinobufagin, 11α-hydroxymarinobufagin, 11α-hydroxytelocinobufagin, 11α-hydroxytelo cinobufagin, Marinosin, 11α,19-dihydroxymarinobufagin, 12β-hydroxycinobufagin, 12β-hydroxytetrahydroresibufogenin 3-sulfate, 15-hydroxybufalin, 16-desacetyl-19-oxocinobufotalin, 16-desacetylcinobufaginol, 19-hydroxybufalin, 19-hydroxybufalin 3-suberoyl-L-3-methylhistidine ester, 19-hydroxybufalin 3-suberoyl-L-histidine ester, 19,hydroxycinobufotalin, 19-hydroxytelocinobufagin, 19-hydroxytelocinobufagin, 19-oxobufalin, 19-oxocinobufagin, 19-oxocinobufotalin, 19-oxodesacetylcinobufagin, 20,21-epoxyresibufagin, 20R,21-epoxyresibufogenin, 20S,21-epoxyresibufogenin, 1,2,3,4-tetrahydro-6-hydroxy-carboline, 2-methyl-6-hydroxy-1,2,3,4-tetrahydro-β-carboline, 3-((N-azelayl argininyl) marinobufagin), 3-(N-adipoyl argininyl) marinobufagin, 3-(N-azelayl argininyl)-bufalin, 3-(N-dodecadienoyl argininyl) marinobufagin, 3-(N-glutaryl argininyl)-marinobufagin, 3-(N-pimeloyl argininyl) marinobufagin, 3-(N-pimeloyl argininyl) telocinobufagin, 3-(N-sebacyl argininyl) marinobufagin, 3-(N-sebacyl argininyl) telocinobufagin, 3-(N-sebacyl argininyl)-bufalin, 3-(N-suberoyl argininyl) hellebrigenin, 4-amido-3-hydroxymethyl-cyclooctylamidezotetra-alpha-furanone 5-hydroxytryptophan, 5-hydroxy-1H-indole-3-carbaldehyde, 5-hydroxyindoleacetic acid, 5-hydroxytryptophol, 5-methoxytryptophol, 5-methoxyindoleacetic acid, bufothionine, dehydrobufotenine, dehydrobufoteninehydrobromide, O-methylnordehydrobufotenine, 6-hydroxy-1-oxo-3,4-dihydro-β-carboline, Marinobufotoxin, Telocinobufatoxin, Bufalitoxin, 3-(N-undecadienoyl argininyl) marinobufagin, 3-beta-formyloxyresibufogenin, 3-O-formyl-20R,21-epoxy resibufogenin, 3-oxo-20S,21-epoxyresibufogenin, 4β-hydroxybufalin, 5Z,9Z-3-(1-hydroxybutyl)-5-propylindolizidine, 6-alpha-hydroxycinobufagin, 6Z,10E-4,6-di(pent-4-enyl) quinolizidine, adenine, arenobufagin, arenobufagin 3-sulfate, arenobufagin hemisuberate, arenobufotoxin, argentinogenin, argininesuberoyl marinobufagenin, azelayl arginine, bacagin, BLP1, BLP2, BLP3, bufalin, bufalon, bufotalone, 5-hydroxycinobufaginol, arenobufagin-3-hemisuberate, cinobufotalin-3-hemisuberate,desacetylbufotalin, bufotalin-3-suberoyl-arginineester, bufalin-3-suberoyl-arginineester, cinobufagin-3-suberoyl-arginineester, Resibufogenin-3-suberoyl-arginineester, 11-Hydroxyhellebrigenol, 5-Hydroxygambufotalin, 5,11-Hydroxyresibufaginol, Bufarenogin, ψ-Bufarenogin, 5-Hydroxyarenobufagin, Hellebrigenol-3-X1, Hellebrigenol-3-X2, Hellebrigenol-3-X3,12-Hydroxyresibufogenin, 5-Hydroxybufotalin, 12, oxobufalin, 3-Acetylresibufogenin,19-oxoresibufagin, 1-Hydroxybufalin, Bufalin-3β-acrylic ester, Telocinobufagin-3-hemisuberate, 3-Oxo-cinobufotalin, Hellebrigenin-3-hemisuberate, 1-Hydroxyarenobufagin, 16-Hydroxytelocinobufagin, Cinobufotalin-3-azelaoyl-arginineester, Cinobufagin-3-succinoyl-arginineester, Bufotalin-3-succinoyl-arginineester, Hellebrigenin-3-suberoyl-arginineester, Bufalin-3-succinoyl-arginineester Telocinobufagin-3-suberoyl-arginineester, Bufotalin-3-pimeloyl-arginineester, 19-Oxocinobufagin-3-adipoyl-arginineester, Telocinobufagin-3-succinoyl-arginineester, Bufalin-3-pim-1, Bufarenogin-3-suberoyl-arginineester, Desacetylcinobufaginol-3-suberoyl-arginineester, Arenobufagin-3-suberoyl-arginineester, Cinobufagin-3-hemisuberate, 12-hydroxybufalin, 16-Hydroxyhellebrigenin, Argentinogenin-3-hemisuberate, Argentinogenin-iso, 3-Oxoargentinogenin, 3-Oxoarenobufagin, Hellebrigenol-3-sulfate, Hellebrigenin-3-sulfate, 19-Hydroxybufalin-3-sulfate, Telocinobufagin-3-sulfate, Arenobufagin-16-acetyl, Resibufogenin-3-formly, 5β,12β-Dihydroxycinobufagin, 16β-Acetoxybufarenogin, 11α,12β-Dihydroxybufalin, 20S,21-Epoxymarinobufagin, 20S,21-Epoxymarinobufagin-3-Acetyl, Arenobufagin-3-sulfate, Bufalin-3-adipoyl-arginineester, Bufalin-3-pimeloyl-arginineester, Bufotalin-3-sulfate, Cinobufagin-3-adipoyl-arginineester, Cinobufagin-3-glut-2, Cinobufagin-3-pimeloyl-arginineester, Cinobufagin-3-sulfate, Cinobufotalin-3-suberoyl-arginineester, Desacetylcinobufagin-3-succinoyl-arginineester, Gambufotalin-3-adipoyl-arginineester, Gambufotalin-3-pimeloyl-arginineester, Gambufotalin-3-suberoyl-arginineester, Gambufotalin-3-succinoyl-arginineester, Gambufotalin-3-sulfate, Marinobufagin-3-glut-2, Marinobufagin-3-pimeloyl-arginineester, Marinobufagin-3-glut-2, Marinobufagin-3-suberoyl-arginineester, Marinobufagin-3-succinoyl-arginineester, Resibufogenin-3-succinoyl-arginineester, Telocinobufagin-3-glut-2,12-hydroxycinobufagin, resibufogenin-3-hemisuberate, bufalin-3-hemisuberate, cinobufagin-3-suc-1, Desacetylcinobufagin-3-suc-1, gambufotalin-3-hemisuberate, bufalin 3-adipoyl-L-arginine ester, bufalin 3-pimeloyl-L-arginine ester, bufalin 3-suberoyl-L-histidine ester, bufalin 3-succinoyl-L-arginine ester, bufalin hemisuberate, bufalin-3-sulfate, bufogargarizin A, bufogargarizin B, bufogargarizin C, bufogargarizin D, bufogenin, bufotalin, bufotalin 3-suberoyl-L-1-methylhistidine ester, bufotalin 3-suberoyl-L-3-methylhistidine ester, Vulgarobufotoxin, bufotalin 3-succinoylarginine ester(I), bufotalin 3-sulfate, bufotalinin, bufotalone, bufotoxin, caffeine, cholesterol, cinabufotalitoxin, cinabufotoxin, cinobufagin, cinobufagin 3-adipoyl-L-arginine ester, cinobufagin 3-glutaryl-L-arginine ester, cinobufagin 3-pimeloyl-L-arginine ester, Cinobufotoxin, cinobufagin 3-succinoyl-L-arginine ester, cinobufagin 3-sulfate, cinobufagin hemisuberate, cinobufaginol, cinobufotalin cinobufotalin 3-suberoyl-L-arginine ester, cyclo(pro-gly)dipeptide, desacetylcinobufagin, desacetylcinobufagin 3-hemisuccinate, desacetylcinobufagin 3-succinyl-L-arginine, desacetylcinobufotalin, Daigredorigenin, dopamine, epinephrine, Eritadenine, gamabufotalin, gamabufotalin 3-adipoyl-L-arginine ester, gamabufotalin 3-pimeloyl-L-arginine ester, Gamabufotalitoxin, gamabufotalin 3-succinoyl-L-arginine ester, gamabufotalin 3-sulfate, gamabufotalin hemisuberoate, gamabufotaliniol, gamma sitosterol, guanine, hellebrigenin, Hellebritoxin, hellebrigenol, hypoxanthine, indoleacetic acid, leucine, marinobufagin, marinobufagin 3-glytaryl-L-arginine ester, marinobufagin 3-pimeloyl-L-arginine ester, marinobufagin 3-suberoyl-L-arginine ester, marinobufagin 3-suberoyl-L-glutamine ester, marinobufagin 3-succinoyl-L-arginine ester, marinobufagin 3-sulfate, marinoic acid, morphine, N-(2-(5-hydroxy-1H-indol-3-yl)ethyl)-N-methylformamide, nicotinamide, nicotinic acid, norepinephrine, palmitic acid cholesterol ester, precoccinelline, resibufagin, resibufagin 3-sulfate, resibufaginol, resibufogenin, resibufagenin, resibufogenin 3-suberoyl-L-arginine ester, resibufogenin 3-succinoyl-L-arginine ester, resibufogenin hemisuberate, resibufotoxin, sebacyl arginine, shepherdine, suberic acid, succinic acid, telocinobufagin, Telocinobufogenin, telocinobufagin 3-glutaryl-L-arginine ester, telocinobufagin 3-suberoyl-L-arginine ester, telocinobufagin 3-suberoyl-L-glutamine ester, thiamethoxam, thymine, tricaine methanesulfonate, uracil, valine, Bombinakinin M, Maximin 1, Maximin, 2, Maximin 3, Maximin 4, Maximin 5, Maximin 6, Maximin 7, Maximin 8, Maximin 9, Maximin 10, Maximin H1, Maximin H2 Maximin H3, Maximin H4, Maximin H5, Maximin H6, Maximin H7, Maximin H8, Maximin H9, Maximin H10, Maximin H11, Maximin H12, Maximin H13, Maximin H14, Maximin H15, Maximin H16, Bradykinin, (Thr(6))-bradykinin, Bombinakinin-GAP, sleep-inducing factor (SIF), xanthine and mixtures thereof.

A pharmaceutical formulation of the disclosure may comprise a composition of the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. The disclosures of US 2018/0221396 A1 and US 2019/0142851 A1 are incorporated herein by reference. According to this disclosure composition containing two purified toad secretion tryptamines as discussed above may be used in place of a “first purified psilocybin derivative” in the compositions described in US 2018/0221396 A1 and US 2019/0142851 A1. Accordingly, the disclosure provides a pharmaceutical formulation comprising as a first component: a composition containing two purified toad secretion tryptamines according to this disclosure and as a second component selected from (a) a purified psilocybin derivative, (b) one or two purified cannabinoids and (c) a purified terpene; with the rest being at least one suitable pharmaceutical excipient or at least one other adjuvant, as discussed below. Such a composition may be a pharmaceutical composition wherein the components are present individually in therapeutic effective amounts or by combination in a therapeutically effective amount to treat a disease, disorder or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed preferred embodiments, incorporated here by reference. Some exemplary serotonergic drugs include the following molecules: 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha,N-Dimethyl-5-methoxy-T. For additional information regarding these compounds See Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof.

Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed preferred embodiments, incorporated here by reference. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl]dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed preferred embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (.DELTA.8-THC), Delta-8-tetrahydrocannabinolic acid (.DELTA.8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed preferred embodiments, incorporated here by reference. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, alpha-caryophyllene, beta-caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, beta-myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, beta-caryophyllene, cedrene, cymene, elemene, eucalyptol, eudesmol, farnesene, fenchol, geraniol, guaiacol, humulene, isoborneol, limonene, linalool, menthol, beta-myrcene, nerolidol, ocimene, phellandrene, phytol, pinene, pulegone, sabinene, terpineol, terpinolene, or valencene.

The compositions and formulations disclosed herein are products of human ingenuity, i.e., made by humans and substantially different from how they are found in nature. The disclosed compositions and formulations can be distinguished from naturally occurring forms by comparing the cellular pharmacology of the disclosed formulations with that of naturally occurring forms. The disclosed compositions and formulations can be distinguished from naturally occurring forms by comparing the molar ratios of compounds within the disclosed formulations with those found in nature. The disclosed compositions and formulations can also be distinguished from naturally occurring forms by comparing the molar ratios of compounds within the disclosed formulations with reference compounds that are present alongside the said compounds when those compounds are found in nature.

At the time of this disclosure, toad secretion tryptamines were only available within complex mixtures comprising other secretion matter. All data indicate that the presence and amounts of psychoactive compounds within naturally occurring samples are considered highly variable. Toad secretions and extracts often do not provide the same physical, cellular, and/or clinical properties as formulations made by combining particular compounds of known purity. In contrast, each of the compositions disclosed herein differs from previously known compositions in significant ways. For example, in the disclosed compositions and formulations, the ratio of toad secretion compounds (e.g., the first purified toad secretion tryptamines to the second toad secretion tryptamine) or the ratio of an toad secretion compound to a naturally occurring reference compound (e.g., cellulose, ligin, chlorophyll, etc) differs from those previously disclosed or otherwise naturally occurring. This disclosure provides compositions and formulations made with known amounts of known compounds, including known amounts of toad secretion compounds. Such formulations allow for administering consistent amounts of toad secretion compounds, which provides the user or subject with consistent and reliable effects.

A “purified” compound is a compound that is in a pure chemical form, not as it existed in nature, i.e., in a toad secretion. A “purified” compound is a higher purity (% purity) than is found in nature. The compound may be extracted and purified by means known in the art. For example, a toad secretion tryptamine may have been chromatographed, for example by gas chromatography, liquid chromatography (e.g., LC, HPLC, etc.), flush column chromatography, etc. or subject to crystallization, distillation, or sublimation. A compound may be purified by two or more purifications steps using those techniques or a composition of those techniques. A purified compound is a compound that is 80-100% pure, 90-100% pure or 95-100% pure. Within the context of this disclosure, the term “purified” means separated from other materials, such as plant or fungal material, e.g., protein, chitin, cellulose, or water. A purified a compound is substantially free of other materials. For example, a purified a compound is substantially free from a second tryptamine compound; substantially free from histidine; substantially free from a biological material, such as mold, fungus, plant mater, or bacteria; or substantially free from a different unwanted compound, e.g., a compound correlated with unwanted side effects.

The disclosure also provides methods of regulating the activity of a neurotransmitter receptor by administering to a person in need thereof an effective dose of a composition of the disclosure or administering to a person in need thereof a formulation of the disclosure. the methods disclosed herein comprise treating a psychological disorder, e.g., an anxiety disorder, a compulsive disorder (e.g., an addiction), a depressive disorder, etc., with a disclosed composition or a disclosed pharmaceutical formulation. In one embodiment, the methods disclosed herein comprise treating a psychological disorder, e.g., an anxiety disorder, a compulsive disorder (e.g., and addiction), a depressive disorder, etc., by administering to a subject in need of treatment one or more of the compositions disclosed herein and a neurotransmitter activity modulator, e.g., a serotonergic drug, a dopaminergic drug, etc. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

### REFERENCES

1. Schmeda-Hirschmann G, Quispe C, Theoduloz C, de Sousa P T, Parizotto C. Antiproliferative activity and new argininyl bufadienolide esters from the “cururu” toad Rhinella (Bufo) schneideri. Journal of Ethnopharmacology. 2014; 155(2):1076-1085. doi:10.1016/j.jep.2014.06.025
2. Medeiros D S S de, Rego T B, Santos A P de A dos, et al. Biochemical and Biological Profile of Parotoid Secretion of the Amazonian Rhinella marina (Anura: Bufonidae). BioMed Research International. doi:10.1155/2019/2492315
3. Uthaug M V, Lancelotta R, van Oorsouw K, et al. A single inhalation of vapor from dried toad secretion containing 5-methoxy-N,N-dimethyltryptamine (5-MeO-DMT) in a naturalistic setting is related to sustained enhancement of satisfaction with life, mindfulness-related capacities, and a decrement of psychopathological symptoms. Psychopharmacology. April 2019. doi:10.1007/s00213-019-05236-w
4. Erspamer V, Vitali T, Roseghini M, Cei J M. 5-Methoxy- and 5-Hydroxyindoles in the skin of Bufo alvarius. Biochemical Pharmacology. 1967; 16(7):1149-1164. doi:10.1016/0006-2952(67)90147-5
5. Pachter I J, Zacharias D E, Ribeiro O. Indole Alkaloids of Acer saccharinum (the Silver Maple), Dictyoloma incanescens, Piptadenia colubrina, and Mimosa hostilis. The Journal of Organic Chemistry. 1959; 24(9):1285-1287. doi:10.1021/jo01091a032
6. Krebs-Thomson K, Ruiz E M, Masten V, Buell M, Geyer M A. The roles of 5-HT1A and 5-HT2 receptors in the effects of 5-MeO-DMT on locomotor activity and prepulse inhibition in rats. Psychopharmacology. 2006; 189(3):319-329. doi:10.1007/s00213-006-0566-1
7. Weil A T, Davis W. Bufo alverius: A potent hallucinogen of animal origin. Journal of Ethnopharmacology. 1994; 41:1-8.
8. Winkelman M. Psychedelics as Medicines for Substance Abuse Rehabilitation: Evaluating Treatments with LSD, Peyote, Ibogaine and Ayahuasca. Current Drug Abuse Reviews. 2014; 7(2):101-116. PMID:25563446.
9. Manske R H F. A Synthesis of the Methyltryptamines and Some Derivatives. Can J Res. 1931; 5(5):592-600. doi:10.1139/cjr31-097
10. Szára St. Dimethyltryptamin: Its metabolism in man; the relation of its psychotic effect to the serotonin metabolism. Experientia. 1956; 12(11):441-442. doi:10.1007/BF02157378
11. Handovsky H. Ein alkaloid im gifte von Bufo vulgaris. Naunyn-Schmiedeberg's Archives of Pharmacology. 1920; 86(1):138-158. doi:10.1007/BF01864237
12. Wieland H, Konz W, Mittasch H. Die Konstitution von Bufotenin und Bufotenidin. Über Kröten-Giftstoffe. VII. Justus Liebigs Annalen der Chemie. 1934; 513(1):1-25. doi:10.1002/jlac.19345130102
13. Hoshino T, Shimodaira K. Synthese des Bufotenins und über 3-Methyl-3-β-oxyäthyl-indolenin. Synthesen in der Indol-Gruppe. XIV. Justus Liebigs Annalen der Chemie. 1935; 520(1):19-30. doi:10.1002/jlac.19355200104
14, Axelsson S, Björklund A, Seiler N. Identification of bufotenin in toad brain by chromatography and mass spectrometry of its dans-derivative. Life Sciences. 1971; 10(13, Part 1):745-749. doi:10.1016/0024-3205(71)90118-4
15. Kärkkäinen J, Räisänen M, Naukkarinen H, Spoov J, Rimon R. Urinary excretion of free bufotenin by psychiatric patients. Biological Psychiatry. 1988; 24(4):441-446. doi:10.1016/0006-3223(88)90182-5
16. Narasimhachari N, Himwich H E. GC-MS identification of bufotenin in urine samples from patients with schizophrenia or infantile autism. Life Sciences. 1973; 12(10, Part 2):475-478. doi:10.1016/0024-3205(73)90335-4
17. Takeda N, Ikeda R, Ohba K, Kondo M. Bufotenine reconsidered as a diagnostic indicator of psychiatric disorders. Neuroreport. 1995; 6(17):2378-2380. http://europepmc.org/abstract/med/8747157. Accessed Mar. 2, 2019.
18. Egan C, Grinde E, Dupre A, et al. Agonist high and low affinity state ratios predict drug intrinsic activity and a revised Ternary complex mechanism at serotonin 5-HT2A and 5-HT2C receptors. Synapse. 2000; 35(2):144-150. doi:10.1002/(SICI)1098-2396(200002)35:2<144::AID-SYN7>3.0.CO; 2-K
19. Peroutka S J, Ison P J, Liu D U, Barrett R W. Artifactual High-Affinity and Saturable Binding of [3H]5-Hydroxytryptamine Induced by Radioligand Oxidation. Journal of Neurochemistry. 1986; 47(1):38-45. doi:10.1111/j.1471-4159.1986.tb02828.x
20. Milburn C M, Peroutka S J. Characterization of [3H]Quipazine Binding to 5-Hydroxytryptamine3 Receptors in Rat Brain Membranes. Journal of Neurochemistry. 1989; 52(6):1787-1792. doi:10.1111/j.1471-4159.1989.tb07258.x
21. Gozlan H, El S M, Pichat L, Glowinski J, Hamon M. Identification of presynaptic serotonin autoreceptors using a new ligand: 3H-PAT. Nature. 1983; 305(5930):140-142. doi:10.1038/305140a0
22. Davis A K, So S, Lancelotta R, Barsuglia J P, Griffiths R R. 5-methoxy-N,N-dimethyltryptamine (5-MeO-DMT) used in a naturalistic group setting is associated with unintended improvements in depression and anxiety. The American Journal of Drug and Alcohol Abuse. 2019; 0(0):1-9. doi:10.1080/00952990.2018.1545024
23. Cameron L P, Benson C J, Dunlap L E, Olson D E. Effects of N,N-Dimethyltryptamine on Rat Behaviors Relevant to Anxiety and Depression. ACS Chem Neurosci. 2018; 9(7):1582-1590. doi:10.1021/acschemneuro.8b00134

1. A composition comprising two purified toad secretion tryptamines chosen from the following 5-MeO-DMT, 5-MeO-NMT, 5-Methoxytryptamine, bufobutanoic Acid, bufobutarginine, bufoserotonin A, bufoserotonin B, bufoserotonin C, bufotenidine, bufotenin, bufotenin Oxide, bufotenine-O-Sulphate, bufoviridine, dET, dMT, n-Acetylserotonin, n′-Formylserotonin, n-Methylserotonin, o-Methylbufoviridine, serotonin, tryptamine, and bufopyramide or the salts of these toad secretion tryptamines.

2. A composition of claim 1, wherein the molar ratio of the first purified toad secretion tryptamine to the second purified toad secretion tryptamine in the composition is from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1, or about 1:1.

3. A formulation comprising a composition of claim 1 and an excipient.

4. A pharmaceutical formulation comprising a composition of claim 1 and a pharmaceutically acceptable excipient, wherein the first purified toad secretion tryptamine and the second purified toad secretion tryptamine are each present in a therapeutically effective amount.

5. A pharmaceutical composition of claim 4 further comprising a therapeutically effective amount of a serotonergic drug, a purified psilocybin derivative, a purified cannabinoid, or a purified terpene.

6. A pharmaceutical composition of claim 4, wherein the therapeutically effective amount of each tryptamine separately ranges from about 0.5 mg-about 200 mg, about 1 mg-about 100 mg, about 2 mg-about 50 mg, about 5 mg-about 25 mg, or 25 mg.

7. A method of regulating the activity of a neurotransmitter receptor comprising the step of administering to a person in need thereof an effective dose of a composition of claim 1.

8. A method of regulating the activity of a neurotransmitter receptor comprising the step of administering to a person in need thereof a pharmaceutical formulation of claim 4.

9. A method of treating a psychological disorder, a compulsive disorder, or a depressive disorder comprising the step of administering to a person in need thereof an effective dose of a composition of claim 1.

10. A method of treating a psychological disorder, a compulsive disorder, or a depressive disorder comprising the step of administering to a person in need thereof a pharmaceutical formulation of claim 4.

11. A formulation comprising a composition of claim 2 and an excipient.

12. A pharmaceutical formulation comprising a composition of claim 2 and a pharmaceutically acceptable excipient, wherein the first purified toad secretion tryptamine and the second purified toad secretion tryptamine are each present in a therapeutically effective amount.

13. A pharmaceutical composition of claim 12 further comprising a therapeutically effective amount of a serotonergic drug, a purified psilocybin derivative, a purified cannabinoid, or a purified terpene.

14. A pharmaceutical composition of claim 5, wherein the therapeutically effective amount of each tryptamine separately ranges from about 0.5 mg-about 200 mg, about 1 mg-about 100 mg, about 2 mg-about 50 mg, about 5 mg-about 25 mg, or 25 mg.

15. A method of regulating the activity of a neurotransmitter receptor comprising the step of administering to a person in need thereof an effective dose of a formulation of claim 3.

16. A method of regulating the activity of a neurotransmitter receptor comprising the step of administering to a person in need thereof a pharmaceutical formulation of claim 5.

17. A method of treating a psychological disorder, a compulsive disorder, or a depressive disorder comprising the step of administering to a person in need thereof an effective dose of a composition of claim 2.

18. A method of treating a psychological disorder, a compulsive disorder, or a depressive disorder comprising the step of administering to a person in need thereof an effective dose of a formulation of claim 3.

19. A method of treating a psychological disorder, a compulsive disorder, or a depressive disorder comprising the step of administering to a person in need thereof a pharmaceutical formulation of claim 5.

20. A method of treating a psychological disorder, a compulsive disorder, or a depressive disorder comprising the step of administering to a person in need thereof a pharmaceutical formulation of claim 6.
